(19) United States
(12) Patent Application Publication
(10) Pub. No.: US 2011/0244030 A1
(43) Pub. Date: Oct. 6, 2011

(54) TOPICAL FOAMING COMPOSITON AND METHOD OF APPLICATION

(76) Inventor: Roc Lebel, St-Helene de Chester (CA)

(21) Appl. No.: 13/133,959

(22) PCT Filed: Dec. 9, 2009
(86) PCT No.: PCT/CA09/01804

§ 371 Date: Jun. 10, 2011

(30) Foreign Application Priority Data
Dec. 10, 2008 (CA) 2646932

(51) Int. Cl. A61K 36/886; A61K 47/46; A61K 8/92; A61Q 17/04; A61K 36/28; A61K 8/97; A61K 9/127; A61P 31/10

(52) U.S. Cl. 424/450; 514/783; 424/59; 424/60; 424/744; 424/737; 424/776

(57) ABSTRACT

There is provided a topical foaming composition for the uniform distribution of formulations without drizzle formation. In accordance with the present invention, the topical composition contains a foaming agent and a pharmaceutically acceptable topical carrier. In accordance with the present invention, the composition may contain at least one active agent incorporated into the topical foaming composition. In accordance with a method of the present invention, the composition is capable of forming a stable foam in the present of a gas and the foam produced therefrom is topically applied on a subject in need thereof.

### BACKGROUND

(a) Field

The subject matter disclosed generally relates to a topical foaming composition and methods of using the same. More specifically, the subject matter generally relates to a topical foaming composition containing an active agent, a foaming agent, and an acceptable pharmaceutical topical carrier, and methods of using the same without drizzle formation.

(b) Related Prior Art

The known method currently used for the topical application of an analgesic agent consists in applying the analgesic agent on a patient with the help of a balm or ointment. The advantage of this method is that it allows dispersion of the epidermis of a uniform quantity of a medicine or drug, while also providing a manual massage action. The known disadvantage of this method is that the medicine or drug must be diluted in large volumes of liquid or gel. The liquid or gel is a material that by the nature of its volume requires a large container.

To overcome this disadvantage, U.S. Pat. No. 7,060,253 to Mundschenk describes a method using an aerosol for the distribution of a liquid or a product capable for forming a foam on the epidermis in a uniform layer. However, this method has the disadvantage of producing microparticles or fine droplets during activation of the aerosol. These particles or droplets in suspension in the air may penetrate the respiratory airways and provoke adverse reactions. It should be noted that in Canada, the use of an aerosol for the application of ointments, liniments, balms and analgesic cream is prohibited.

### SUMMARY

In a first embodiment, there is disclosed a topical foaming composition for admixing with an active agent comprising a foaming agent, and a pharmaceutically acceptable carrier.

The topical foaming composition may comprise an effective amount of at least one active agent.

The foaming agent may be a cationic surfactant, an anionic surfactant, an amphoteric surfactant, a non-ionic surfactant or combination thereof. The foaming agent may be at least one of propylene glycol, Triaminox LO, or combination thereof.

The active agent may be an analgesic drug. The analgesic drug may be methyl salicilate, salicylic acid, aspirin, indometacin, diclofenac, ibuprofene, ketoprofen, naproxen, ketorolac, mefenamic acid, piroxicam, meloxicam, celecoxib, rofecoxib, parecoxib, etocoxib, nimesulide, codein, folic acid, chamomile extract, willow extract, humulus lulupus extract, callophyllum extract or combinations thereof.

The active agent may be an irritating pharmaceutical ingredients. The irritating pharmaceutical ingredient may be allyle isothiocyanate, ammoniaque officinale, methyl nicotinate, methyl salicilate (wintergreen), oil of turpentine, histamine dichlorhydrate, eucalyptus oil, eucalyptol oil, thymol, clove oil, or combination thereof.

The active agent may be a nanoparticle pharmaceutical ingredient. The nanoparticle pharmaceutical ingredient may be a silver particle, a drug delivery particle, or combinations thereof. The drug delivery particle may be a micelle, a reverse micelle, a liposome, or combinations thereof.

The active agent may be a topical analgesic compound. The topical analgesic compound may be a capsaicinoid, resiniferatoxin, cinnamaldehyde, menthol, eucalyptol, camphor, norcamphor, or combination thereof. The capsaicinoid may be capsaicin, dihydrocapsaicin, nordihydrocapsaicin, homodihydro capsaicin, homocapsaicin, nonivamide, or combination thereof.

The active agent may be of synthetic or natural origin.

The foaming agent may be of synthetic or natural origin.

The composition may contain an essential oil. The essential oil may be argan oil, cypress oil, chamomile oil, aniba rosaeodora oil, lavender oil, tea tree oil, pine tree oil, eucalyptol oil, eucalyptus oil, birch oil, peppermint oil, ylang-ylang oil, cymbopogon martinii oil, sweet almond oil, olive oil, or combination thereof.

The composition may contain a vegetal oil. The vegetal oil may be olive oil, canola oil, corn oil, sunflower oil, sesame oil, castor oil, safflower oil, argan oil, linseed oil, grape seed oil, avocado oil or combination thereof.

The composition may contain an alcool. The alcool may be ethanol, isopropanol, propanol, methanol, or combination thereof.

The composition may contain a moisturizer. The moisturizer may be stearic acid: myrestyl alcohol, white petrolatum, glycerin, lanolin, hydrogenated polydecene, cetearyl alcohol, aloe vera gel, or combination thereof.

The composition may contain a sun screening compound. The sun screening compound may be octyl methoxycinnamate (octinoxate), homosalate, oxibenzone, avobenzone, or combination thereof. The composition may also contain marine collagen, titanium dioxide, zinc oxide or combination thereof.

The composition may contain an antiseptic agent. The antiseptic agent may be benzalkonium chloride, chlorhexidine gluconate, glucono delta-lactone, a paraben compound, benzoic acid, imidazolidinyl urea, a quaternary ammonium compound, octenidine dihydrochloride, zinc oxide, citric acid or combination thereof.

The composition may contain a vitamin. The vitamin may be vitamin A, biotin, vitamin E, vitamin C, vitamin D, bioflavonoids (vitamin P) or combination thereof.

The composition may contain a mineral. The mineral may be zinc, sodium, potassium, selenium, manganese, copper, calcium, silica or combination thereof.

The composition may contain a plant extract. The plant extract may be Echinacea, chamomile extract, lavender extract, birch extract, salvia extract, humulus lulupus (common hop) extract, wheat extract, wheat germ extract, liquorice extract, Melaleuca quinquenervia (Niaouli) extract, Gaultheria procumbens extract, geranium extract, oenothera extract, argan extract, althaea officinalis extract, aloe vera extract, jojoba extract, ginkgo biloba extract, green tea extract, olive extract, peppermint extract, eucalyptus extract, hypericum extract, willow extract or combination thereof.

The composition may contain a seed extract. The seed extract may be apricot seed extract, grapefruit seed extract, orange seed extract, lemon seed extract, lime seed extract, melon seed extract, Shea butter, or combination thereof.

In a second embodiment there is disclosed a method of topically applying a foam comprising:
topically applying on a subject a foam produced from foaming a topical foaming composition with a gas.

The foaming may be effected with a metered dose pump.

The topically applying on a subject may be with a hand, an absorbent pad, or a brush.

The following terms are defined below.

The terms “Carriers” or “vehicles” are intended to mean carrier materials suitable for compound administration and include any such material known in the art such as, for example, any liquid, lotion, gel, solvent, liquid diluent, solubilizer, or the like, which is non-toxic and which does not interact with any components of the composition in a deleterious manner.

The term “epidermis” is intended to mean the outer layer of the skin, composed of terminally differentiated stratified squamous epithelium, acting as the body's major barrier against an inhospitable environment. It is the thinnest on the eyelids at 0.05 mm and the thickest on the palms and soles at 1.5 mm.

The term “gas” is intended to mean the state of matter distinguished from the solid and the liquid by: relatively low density and viscosity; relatively great expansion and contraction with changes in pressure and temperature; the ability to diffuse readily; and the spontaneous tendency to become distributed uniformly throughout any container. A gas may be composed of atoms, molecules or larger particles, that may be homogenous (comprising only one type of atoms, molecules, or particles) for example pure oxygen gas, or heterogenous (comprising more than one type of atoms, molecules or particles), for example air.

The term “natural” is intended to mean existing in or produced by nature; not artificial or imitation. For example, when relating to a product of natural origin, it may have been produced from a plant (e.g. a plant extract), a mineral or from the sea (e.g. sea salt).

The term “synthetic” is intended to mean not of natural origin; prepared or made artificially. For example, when relating to a product of synthetic origin, it may have been produced by man (e.g. a pair of shoe, synthetic polyester fiber, etc).

The term “pharmaceutically acceptable carrier” is intended to mean a preservative solution, a saline solution, an isotonic (about 0.9%) saline solution, or about a 5% albumin solution, suspension, sterile water, phosphate buffered saline, and the like. Other buffering agents, dispersing agents, and inert non-toxic substances suitable for delivery to a patient may be included in the compositions of the present invention. The compositions may be solutions, suspensions or any appropriate formulation suitable for administration, and are typically sterile and free of undesirable particulate matter. The compositions may be sterilized by conventional sterilization techniques.

The term “topical” is intended to mean that the composition or medication is suitable for application on body surfaces such as the skin or mucous membranes such as the vagina, anus, throat, eyes and ears.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### DETAILED DESCRIPTION OF THE PREFERRED EMBODIMENTS

In embodiments there are disclosed topical foaming compositions comprising an effective amount of at least one active agent, and a foaming agent, in association with a pharmaceutically acceptable topical carrier.

The formulations may contain a foaming agent. Foaming agents may be a cationic surfactant, an anionic surfactant, an amphoteric surfactant, or a non-ionic surfactant. Preferably, the foaming agent is a non-ionic surfactant, such as the product sold under the trade name of TRIAMINOX LO by Canada Colors and Chemicals Ltd., which is an alkyl dimethyl amine oxide 30%. Other preferred foaming agents include propylene glycol, and emso 31.

The composition contain at least one active agent. Active agent will vary according to the intended use of the topical foaming composition of the present invention. Example of active agents include but are not limited to:

Analgesic Drugs

The analgesic drugs that may be used in the present invention may be methyl salicilate, salicylic acid, aspirin, indometacin, diclofenac, ibuprofene, ketoprofen, naproxen, ketorolac, mefenamic acid, piroxicam, meloxicam, celecoxib, rofecoxib, parecoxib, etocoxib, nimesulide, codein, and folic acid. The analgesic may also be an extract from chamomile, willow, humulus lulupus (common hop) and callophyllum.

Irritating Pharmaceutical Ingredients

The irritating pharmaceutical ingredients may be Allyl isothiocyanate, an ammoniaque officinale (ammonia water), camphor, capsaicin, menthol, methyl nicotinate, methyl salicylate, essence of turpentine, histamine dichlorohydrate, eucalyptus oil, eucalyptol oil, thymol, or clove oil.

Nanoparticle Pharmaceutical Ingredients

The nanoparticle pharmaceutical ingredient may be silver nanoparticles, which may be used as a antimicrobial. The nanoparticle may also be particles that are used for the delivery of drug particles, such as those used in cancer therapy, the treatment of erectile dysfunction. Nanoparticles used for such usage may be vesicles, which comprise a membrane that is made of lipid, usually but not always phospholipids. The vesicles may be formed as micelles with a monolayer of lipids, or as liposomes, which have a bilayer structure. Micelles usually have a hydrophobic (water repelling) core in which drugs with low solubility may be contained. Reverse micelles, which are micelles with an aqueous core may be used to encompass soluble drugs. Liposomes usually but not always have a core containing an aqueous solution, which is suitable for delivery of drugs with higher solubility. Liposomes may also have a hydrophobic core which may be used for delivery of drugs with low solubility.

Topical Analgesic Compounds

The composition of the present invention may contain as active agent a topical analgesic compound to cause a sensation of warmth, a sensation of coolness or both. Such compounds include capsaicinoid compounds, such as capsaicin, dihydrocapsaicin, nordihydrocapsaicin, homodihydro capsaicin, homocapsaicin, and nonivamide. Compounds of this family are the active component of chili peppers, which are plants belonging to the genus Capsicum.

The topical analgesic also include resiniferatoxin, which is a naturally occurring, ultrapotent capsaicin analog that activates the vanilloid receptor in a subpopulation of primary afferent sensory neurons involved in nociception (the transmission of physiological pain).

Also included is cinnamaldehyde, the primary organic compound in cinnamon, menthol, the primary organic compound found in peppermint or other mint oils, eucalyptol (aka limonene oxide, cineol, cineole), which is obtained from plants of the eucalyptus genus, camphor, extracted from the camphor laurel and norcamphor, where three hydrogens have replaced the methyl groups of camphor.

Essential Oils

The composition of the present invention may include essential oils as active agents. Oil are “essential” in the sense that they carry distinctive scent, or essence, of a plant. Preferred essential oils include but are not limited to argan oil, cypress oil, chamomile oil, oil (bois de rose oil) lavender oil, tea tree oil (melaleuque oil), pine tree oil, eucalyptol oil, eucalyptus oil, birch oil, peppermint oil, ylang-ylang oil, cymbopogon martinii oil (palmarosa oil), sweet almond oil and olive oil.

Vegetable Oils

The composition of the present invention may also include vegetable oils, such as olive oil, canola oil, corn oil, sunflower oil, sesame oil, castor oil, safflower oil, argan oil, linseed oil, grape seed oil, and avocado oil.

Alcools

The composition of the present invention may also contain alcohols, such as ethanol, isopropanol, propanol, and methanol.

Moisturizers

The composition of the present invention may also include a moisturizer to moisturize the epidermis to which it is being applied. Moisturizers include but are not limited to stearic acid, myrestyl alcohol, white petrolatum, glycerin, lanolin, hydrogenated polydecene, cetearyl alcohol and aloe vera gel.

Sun Screening Compounds and Components

The composition of the present invention may be used as sun screens. The compositions will therefore incorporate sun screening compounds such as octyl methoxycinnamate (octinoxate), homosalate, oxibenzone, and avobenzone. The sun screening compositions of the present invention may also comprise marine collagen, titanium dioxide, zinc oxide, citric acid or combination thereof.

Antiseptic Agents

The composition of the present invention may also incorporate antiseptic agents to preserve the composition free of microorganisms, as well as treat epidermis infected or affected with microorganisms, or simply as a preventive measure against infection by microorganisms. The antiseptic agents include but are not limited to benzalkonium chloride, chlorhexidine gluconate, glucono delta-lactone, a paraben compound, benzoic acid, imidazolidinyl urea, a quaternary ammonium compound, and octenidine dihydrochloride.

Vitamins

The composition of the present invention may include vitamins. The vitamins include but are not limited to vitamin A, biotin, vitamin E, vitamin C, vitamin D, bioflavonoids (vitamin P) and combinations thereof.

Mineral

The composition of the present invention may include minerals that include but are not limited to zinc, sodium, potassium, selinium, manganese, copper, silica, and calcium.

Plant and Seed Extracts

The composition of the present invention may include plant extracts, such as Echinacea. It may also include seed extracts. These natural extracts complement the composition of the present invention by optimizing the action of the present invention and providing an esthetic effect on the contacted epidermis.

The plant extract may be from chamomile, lavender, birch, salvia, humulus lulupus (common hop), wheat, wheat germ, liquorice, Melaleuca quinquenervia (Niaouli), Gaultheria procumbens (t hé des bois), geranium, oenothera, argan, althaea officinalis, aloe vera, jojoba, ginkgo biloba, green tea extract, olive, peppermint, eucalyptus, hypericum, and willow.

The seed extract may be from apricot, grapefruit, orange, lemon, lime, and melon. The seed extract may also be Shea butter.

Use of the Composition

In use, the composition is used by contacting an epidermis with a foam produced from foaming a composition with air. Foaming is achieved by introducing air into the composition of the present invention with a pump such as a metered dose pump. Preferably, the metered dose pump capable of producing the foam is a manual pump capable of introducing by the application of pressure, a predetermined volume of air into the formulation. The pump may be, for example, a pump such as that described in U.S. Pat. No. 6,840,408 to Foster et al.

In use, the application of foam may be performed by depositing the foam in one hand, on a absorbent (e.g. cotton) pad or a brush and then proceed to a friction movement to uniformly dispense the foam on the epidermis.

### ALTERNATIVE EMBODIMENTS

The following example illustrates the present invention in a non-limiting manner.

### Example I

A liquid formulation containing a medicine or a drug, and a foaming agent in solution has been prepared. This formulation contain (percentage are expressed in weight/weight):

| | |
|---|---|
| Water | 30 to 70% |
| Triaminox LO* | 1 to 6% |
| Eucalyptol | 1 to 4% |
| Menthol | 1 to 4% |
| Aloe vera extract | 0.5% to 5% |
| Vegetable oil | 5% to 15% |
| Ethanol | 2% to 21.5% |

This formulation was then introduced in a 250 ml bottle closed with a cap comprising a pump capable of producing foam. This pump is the G3-L7 produced by AIRSPRAY INTERNATIONAL INC. Manual activation of the metered pump mounted on the cap, of the bottle; the user is able to produce a quantity of foam of approximately 10 to 25 ml. This foam is then applied by friction on the epidermis to be treated, without any risk of respiratory inhalation. This trial has been very positive.

### Example II

A liquid formulation of a sun screening formulation for children and adults has been prepared. This formulation contain (percentage are expressed in weight/weight):

| | |
|---|---|
| Water | 20 to 60% |
| Triaminox LO* | 1 to 7% |
| Lavander oil | 0.25% to 4% |
| Aloe vera extract | 0.5% to 3% |
| Methyl Salicilate | 0.50% to 4% |
| Marine collage | 0.025% to 3% |
| Argan oil | 0.25% to 5% |
| Plant extract | 0.25% to 30% |
| Vitamins | 0.05% to .50% |
| Minerals | 0.05% to 50% |

This formulation was then introduced in a 250 ml bottle closed with a cap comprising a pump capable of producing foam. This pump is the G3-L7 produced by AIRSPRAY INTERNATIONAL INC. Manual activation of the metered pump mounted on the cap, of the bottle, the user is able to produce a quantity of foam of approximately 10 to 25 ml. This foam is then applied by friction on the epidermis to be treated, without any risk of respiratory inhalation. This trial has been very positive.

### Example III

A liquid formulation of a sun screening formulation for children and adults has been prepared. This formulation contain (percentage are expressed in weight/weight):

| | |
|---|---|
| Water | 30% to 60% |
| Triaminox LO* | 1% to 3% |
| Lavander oil | 0.25% to 4% |
| Octinoxate | 1% to 8% |
| homosalate | 1% to 8% |
| oxibenzone | 1% to 8% |
| avobenzone. | 1% to 8% |
| Marine collage | 0.50% to 5% |
| Plant extract | 1% to 25% |
| Vitamins | 0.10% to 1%% |
| Minerals | 1% to 5% |

This formulation was then introduced in a 250 ml bottle closed with a cap comprising a pump capable of producing foam. This pump is the G3-L7 produced by AIRSPRAY INTERNATIONAL INC. Manual activation of the metered pump mounted on the cap, of the bottle, the user is able to produce a quantity of foam of approximately 10 to 25 ml. This foam is then applied by friction on the epidermis to be treated, without any risk of respiratory inhalation. This trial has been very positive.

### Example IV

A liquid formulation of an antifungal (antiseptic) foam formulation has been prepared. This formulation contains (percentage are expressed in weight/weight):

| | |
|---|---|
| Water | 60% |
| Triaminox LO* | 1 to 6% |
| Propylene glycol | 1% to 15% |
| Lavander oil | 0.25% to 3% |
| Tea tree oil | 0.25% to 5% |
| Pine oil | 0.25% to 3% |
| Benzalkonium chloride | 0.25% to 7% |
| Echinacae | 0.10% to .90% |
| Plant extract | 0.1% to 25% |
| Seed extract | 0.25% to 2% |

This formulation was then introduced in a 250 ml bottle closed with a cap comprising a pump capable of producing foam. This pump is the G3-L7 produced by AIRSPRAY INTERNATIONAL INC. Manual activation of the metered pump mounted on the cap, of the bottle, the user is able to produce a quantity of foam of approximately 10 to 25 ml. This foam is then applied by friction on the epidermis to be treated, without any risk of respiratory inhalation. This trial has been very positive.

### Example V

A liquid formulation of a therapeutic massage foam has been prepared. This formulation contain (percentage are expressed in weight/weight):

| | |
|---|---|
| Water | 5% to 60% |
| Triaminox LO* | 1% to 7% |
| Lavander oil | 0.25% to 3% |
| Peppermint oil | 0.50% to 8% |
| Eucalyptol oil | 0.25% to 5% |
| Birch oil | 0.50% to 7% |
| Olive oil | 0.50% to 5% |
| Plant extract | 0.50% to 7% |
| Seed extract | 0.50% to 10% |

This formulation was then introduced in a 250 ml bottle closed with a cap comprising a pump capable of producing foam. This pump is the G3-L7 produced by AIRSPRAY INTERNATIONAL INC. Manual activation of the metered pump mounted on the cap, of the bottle, the user is able to produce a quantity of foam of approximately 10 to 25 ml. This foam is then applied by friction on the epidermis to be treated, without any risk of respiratory inhalation. This trial has been very positive.

### Example VI

A liquid formulation of a sun screening formulation for children and adults has been prepared. This formulation contains aloe vera gel, Gaultheria procumbens (the des bois), lavender oil, marine collagen, plant extract and minerals, argan oil, vegetal oil, isopropanol, althaea officinalis, chamomile oil, jojoba, apricot seed extract, sesame oil, vitamin E, aniba rosaeodora oil, ylang-ylang tree, tea tree oil, zinc oxide, titanium dioxide, palmarosa oil, bioflavonoids and purified water.

### Example VII

A liquid formulation of a sun screening formulation for children and adults has been prepared. This formulation contains octinoxate, homosalate, oxibenzone, avobenzone, marine collagen, plant extracta and minerals, vitamins, triaminox LO, vegetal oil, isopropanol, shea butter, sunflower oil, ginkgo biloba, glycerin, green tea extract, ecchinacae, citric acid and purified water.

### Example VIII

A liquid formulation of an antifungal (antiseptic) foam formulation has been prepared. This formulation contains lavender oil, tea tree oil, pine oil, benzalkonium chloride, echinacea, plant and seed extract, triaminox LO, vegetal oil, isopropyl alcool, propylene glycol, sage oil, bioflavonoids, purified water.

### Example IX

A liquid foam formulation for therapeutic massage has been prepared. This formulation contains peppermint, eucalyptol, lavender oil, birch oil, olive oil, plant and seed extracts, triaminox LO, vegetal oil, isopropanol, althaea officinalis and purified water.

### Example X

A liquid foam formulation of Collagen and silica has been prepared. This formulation contains silica, marine collagen, triaminox LO, sage oil, cypress oil, mint oil, ginger oil, and purified water.

### Example XI

A liquid foam formulation for treatment of major burns has been prepared. The composition contains lanolin, collagen, vegetal oil, hyperycum extract, triaminox LO, bioflavonoids, lavander oil, camomile oil, Melaleuca quinquenervia (niaouli) extract and purified water.

### Example XII

A liquid foam formulation for treatment of psoriasus and/or ecxema has been prepared. The composition contains argan oil, olive oil, avocado oil, althaea officinalis, oenothera, vegetal oil, triaminox LO, bioflavonoids, chamomile oil, sweet almond oil, lavender oil, geranium extract, wheat germ and purified water.

The embodiments and examples presented herein are illustrative of the general nature of the subject matter claimed and are not limiting. It will be understood by those skilled in the art how these embodiments can be readily modified and/or adapted for various applications and in various ways without departing from the spirit and scope of the subject matter disclosed claimed. The claims hereof are to be understood to include without limitation all alternative embodiments and equivalents of the subject matter hereof. Phrases, words and terms employed herein are illustrative and are not limiting. Where permissible by law, all references cited herein are incorporated by reference in their entirety. It will be appreciated that any aspects of the different embodiments disclosed herein may be combined in a range of possible alternative embodiments, and alternative combinations of features, all of which varied combinations of features are to be understood to form a part of the subject matter claimed.

1. A topical foaming composition for admixing with an active agent comprising:
a foaming agent, and
a pharmaceutically acceptable carrier.

2. The topical foaming composition as claimed in claim 1, further comprising:
an effective amount of at least one active agent.

3. The composition of any one of claims 1-2, wherein said foaming agent is a cationic surfactant, an anionic surfactant, an amphoteric surfactant, a non-ionic surfactant or combination thereof.

4. The composition of claim 3, wherein said foaming agent is at least one of propylene glycol, Triaminox LO, or combination thereof.

5. The composition as claimed in claim 2, wherein said at least one active agent is an analgesic drug.

6. The composition as claimed in claim 5, wherein said analgesic drug is methyl salicilate, salicylic acid, aspirin, indometacin, diclofenac, ibuprofene, ketoprofen, naproxen, ketorolac, mefenamic acid, piroxicam, meloxicam, celecoxib, rofecoxib, parecoxib, etocoxib, nimesulide, codein, folic acid, chamomile extract, willow extract, humulus lulupus extract, callophyllum extract or combinations thereof.

7. The composition as claimed in claim 2, wherein said at least one active agent is an irritating pharmaceutical ingredients.

8. The composition as claimed in claim 7, wherein said irritating pharmaceutical ingredient is allyle isothiocyanate, ammoniaque officinale, methyl nicotinate, methyl salicilate (wintergreen), oil of turpentine, histamine dichlorhydrate, eucalyptus oil, eucalyptol oil, thymol, clove oil, or combination thereof.

9. The composition as claimed in claim 2, wherein said at least one active agent is a nanoparticle pharmaceutical ingredient.

10. The composition as claimed in claim 9, wherein said nanoparticle pharmaceutical ingredient is a silver particle, a drug delivery particle, or combinations thereof.

11. The composition as claimed in claim 10, wherein said drug delivery particle is a micelle, a reverse micelle, a liposome, or combinations thereof.

12. The composition as claimed in claim 2, wherein said at least one active agent is a topical analgesic compound.

13. The composition as claimed in claim 12, wherein said topical analgesic compound is a capsaicinoid, resiniferatoxin, cinnamaldehyde, menthol, eucalyptol, camphor, norcamphor, or combination thereof.

14. The composition as claimed in claim 13, wherein said capsaicinoid is capsaicin, dihydrocapsaicin, nordihydrocapsaicin, homodihydro capsaicin, homocapsaicin, nonivamide, or combination thereof.

15. The composition as claimed in any one of claims 2-14, wherein said active agent is of synthetic or natural origin.

16. The composition as claimed in any one of claims 2-14, wherein said foaming agent is of synthetic or natural origin.

17. The composition as claimed in any one of claims 2-14, further comprising an essential oil.

18. The composition as claimed in claim 17, wherein said essential oil is argan oil, cypress oil, chamomile oil, aniba rosaeodora oil, lavender oil, tea tree oil, pine tree oil, eucalyptol oil, eucalyptus oil, birch oil, peppermint oil, ylang-ylang oil, cymbopogon martini oil, sweet almond oil, olive oil, or combination thereof.

19. The composition as claimed in any one of claims 2-14, further comprising a vegetal oil.

20. The composition as claimed in claim 19, wherein said vegetal oil is olive oil, canola oil, corn oil, sunflower oil, sesame oil, castor oil, safflower oil, argan oil, linseed oil, grape seed oil, avocado oil or combination thereof.

21. The composition as claimed in any one of claims 2-20, further comprising an alcool.

22. The composition as claimed in claim 23, wherein said alcool is ethanol, isopropanol, propanol, methanol, or combination thereof.

23. The composition as claimed in any one of claims 2-20, further comprising a moisturizer.

24. The composition as claimed in claim 23, wherein said moisturizer is stearic acid, myrestyl alcohol, white petrolatum, glycerin, lanolin, hydrogenated polydecene, cetearyl alcohol, aloe vera gel, lanolin or combination thereof.

25. The composition as claimed in any one of claims 2-24, further comprising a sun screening compound.

26. The composition as claimed in claim 25, wherein said sun screening compound is octyl methoxycinnamate (octinoxate), homosalate, oxibenzone, avobenzone, or combination thereof.

27. The composition as claimed in any one of claims 25-26, further comprising marine collagen, titanium dioxide, zinc oxide, citric acid or combination thereof.

28. The composition as claimed in any one of claims 2-27, further comprising an antiseptic agent.

29. The composition as claimed in claim 28, wherein said antiseptic agent is benzalkonium chloride, chlorhexidine gluconate, glucono delta-lactone, a paraben compound, benzoic acid, imidazolidinyl urea, a quaternary ammonium compound, octenidine dihydrochloride, zinc oxide or combination thereof.

30. The composition as claimed in any one of claims 2-29, further comprising a vitamin.

31. The composition as claimed in claim 30, wherein said vitamin is vitamin A, biotin, vitamin E, vitamin C, vitamin D, bioflavonoids (vitamin P) or combination thereof.

32. The composition as claimed in any one of claims 2-31, further comprising a mineral.

33. The composition as claimed in claim 32, wherein said mineral is zinc, sodium, potassium, selenium, manganese, copper, calcium, silica or combination thereof.

34. The composition as claimed in any one of claims 2-33, further comprising a plant extract.

35. The composition as claimed in claim 34, wherein said plant extract is Echinacea, chamomile extract, lavender extract, birch extract, salvia extract, humulus lulupus (common hop) extract, wheat extract, wheat germ extract, liquorice extract, Melaleuca quinquenervia (Niaouli) extract, Gaultheria procumbens extract, geranium extract, oenothera extract, argan extract, althaea officinalis extract, aloe vera extract, jojoba extract, ginkgo biloba extract, green tea extract, olive extract, peppermint extract, eucalyptus extract, hypericum extract, willow extract or combination thereof.

36. The composition as claimed in any one of claims 2-35, further comprising a seed extract.

37. The composition as claimed in claim 36, wherein said seed extract is apricot seed extract, grapefruit seed extract, orange seed extract, lemon seed extract, lime seed extract, melon seed extract, Shea butter, or combination thereof.

38. A method of topically applying a foam comprising:
topically applying on a subject a foam produced from foaming a topical foaming composition as claimed in any one of claims 2-37 with a gas.

39. The method of claim 38, wherein said foaming is effected with a metered dose pump.

40. The method of any one of claims 38-39, wherein said topically applying on a subject is with a hand, an absorbent pad, or a brush.
